(19) United States
(12) Patent Application Publication
(10) Pub. No.: US 2018/0235270 A1
(43) Pub. Date: Aug. 23, 2018

(54) PROBIOTIC BEVERAGES CONTAINING CANNABINODIOL

(71) Applicant: Babak BABAN, Augusta, GA (US)

(72) Inventor: Babak BABAN, Augusta, GA (US)

(21) Appl. No.: 15/898,774

(22) Filed: Feb. 19, 2018

Related U.S. Application Data
(60) Provisional application No. 62/460,929, filed on Feb. 20, 2017.

(51) Int. Cl. A23L 33/135; A61K 31/352; A23L 33/125; A23C 9/123; A61P 1/14

(52) U.S. Cl. CPC A23L 33/135; A61K 31/352; A61P 1/14; A23C 9/123; A23L 33/125

(57) ABSTRACT

Disclosed herein are probiotic beverages containing cannabinodiol. The beverages disclosed herein may be formulated as yogurt drinks, for example, and contain live, active cultures of probiotic microorganisms. The beverages may additionally include prebiotic substances including, but not limited to, inulin to enhance the growth and colonization of the probiotic microorganisms in the intestines. The beverages have anti-inflammatory effects due to the presence of cannabinodiol but are non-narcotic. Further, the beverages serve to enhance gut health due to the presence of probiotics and prebiotics. The beverages are, at a minimum, composed of yogurt, cannabinodiol, Lactobacillus bifidus, and Lactobacillus acidophilus but may contain other components such as vitamins, minerals, other nutrients, and other natural products to provide additional health benefits and/or to improve the flavor, texture, and aroma of the beverages.

### CROSS REFERENCE TO RELATED APPLICATIONS

This application claims priority upon U.S. provisional application Ser. No. 62/460,929, filed Feb. 20, 2017. This application is hereby incorporated by reference in its entirety for all of its teachings.

### BACKGROUND

Mammals are hosts to several hundred different species of gut microorganisms, including bacteria, archaea, yeasts, and protozoa. Due to poor diet, medical treatments such as antibiotic use, and even stress or emotional factors, disruptions to the gut microbiota (known as “dysbiosis”) are increasingly common. Symptoms of dysbiosis range from gastrointestinal upset ranging from gas, bloating, and flatulence to indigestion or incomplete digestion, diarrhea, and constipation, fatigue, amenorrhea, iron deficiency, rosacea and adult acne, poor growth, brittleness, and weakness of the hair and nails, and more.

Meanwhile, inflammation is part of the body's immune response and can be beneficial in response to an injury. In fact, the inflammatory response is required for wound healing and repairing damage to tissue. However, chronic inflammation (i.e., in the absence of any acute stimulus including, but not limited to, a wound or cut, a crush injury, a broken bone, and the like.) is increasingly a problem in modern society and has been linked to autoimmune disorders such as rheumatoid arthritis, gum disease or periodontitis, diseases of the circulatory system including atherosclerosis, allergic conditions such as hay fever, diabetes, and inflammatory bowel diseases such as ulcerative colitis and Crohn's disease. Interest in controlling inflammation is growing in both the medical community and the general population.

Many individuals find some degree of relief from dysbiosis by taking probiotic supplements containing beneficial bacteria. These supplements also occasionally contain prebiotic compounds, or indigestible fibers that provide a favorable environment for the probiotic bacteria to take root. In the case of inflammation, plant-based and natural supplements are gaining popularity (e.g., ginger, turmeric/curcumin, etc.) as a safe and effective means to reduce elimination and find relief from chronic conditions such as those discussed above. Further, dysbiosis and inflammation can be related conditions and are often found in the same individuals. Addressing both problems may require swallowing a large number of pills or capsules each day, which can reduce a subject's compliance with a treatment regimen.

It would be advantageous to have a single product, such as a beverage, to address both dysbiosis and inflammation. Further, it would be advantageous for the beverage to have a satisfactory taste so that the subject is encouraged to consume the beverage on a routine basis, for maximum reduction of inflammation as well as to maintain an optimal level of gut health. The present invention addresses these needs.

### SUMMARY

Disclosed herein are probiotic beverages containing cannabinodiol. The beverages disclosed herein may be formulated as yogurt drinks, for example, and contain live, active cultures of probiotic microorganisms. The beverages may additionally include prebiotic substances including, but not limited to, inulin to enhance the growth and colonization of the probiotic microorganisms in the intestines. The beverages have anti-inflammatory effects due to the presence of cannabinodiol but are non-narcotic. Further, the beverages serve to enhance gut health due to the presence of probiotics and prebiotics. The beverages are, at a minimum, composed of yogurt, cannabinodiol, Lactobacillus bifidus, and Lactobacillus acidophilus but may contain other components such as vitamins, minerals, other nutrients, and other natural products to provide additional health benefits and/or to improve the flavor, texture, and aroma of the beverages.

The advantages of the invention will be set forth in part in the description that follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### DETAILED DESCRIPTION

Before the present compounds, compositions, and/or methods are disclosed and described, it is to be understood that the aspects described below are not limited to specific compounds, synthetic methods, or uses as such may, of course, vary. It is also to be understood that the terminology used herein is for the purpose of describing particular aspects only and is not intended to be limiting.

In this specification and in the claims that follow, reference will be made to a number of terms that shall be defined to have the following meanings:

It must be noted that, as used in the specification and the appended claims, the singular forms “a,” “an,” and “the” include plural referents unless the context clearly dictates otherwise. Thus, for example, reference to “a prebiotic” includes mixtures of two or more such prebiotics, and the like.

“Optional” or “optionally” means that the subsequently described event or circumstance can or cannot occur, and that the description includes instances where the event or circumstance occurs and instances where it does not. For example, the phrase “optionally includes a natural flavor” means that the natural flavor may or may not be present.

Ranges may be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

References in the specification and concluding claims to parts by weight, of a particular element or component in a composition or article, denote the weight relationship between the element or component and any other elements or components in the composition or article for which a part by weight is expressed. Thus, in a composition containing 2 parts by weight of component X and 5 parts by weight of component Y, X and Y are present at a weight ratio of 2:5, and are present in such ratio regardless of whether additional components are contained in the composition.

A weight percent of a component, unless specifically stated to the contrary, is based on the total weight of the formulation or composition in which the component is included.

The term “treat” as used herein is defined as maintaining or reducing the symptoms of a pre-existing condition (e.g., inflammation). The term “prevent” as used herein is defined as eliminating or reducing the likelihood of the occurrence of one or more symptoms of a disease or disorder.

In one aspect, described herein is a yogurt-based beverage. “Yogurt” as used herein is a semisolid, sour-tasting food prepared from milk that has been fermented by added bacteria. In one aspect, the milk is dairy milk. Further in this aspect, the milk is from a cow or goat. Still further in this aspect, the milk can be nonfat milk, lowfat milk, or whole milk. In one aspect, “lowfat” milk is defined as milk containing 1% or 2% fat, by weight. In another aspect, milk solids can be added to the yogurt-based beverage. In still another aspect, cream can be added to the yogurt-based beverage at a point before, during, or after fermentation.

In an alternative aspect, the milk is a non-dairy milk such as, for example, soy milk, almond milk, cashew milk, coconut milk, hemp milk, oat milk, rice milk, flax milk, hazelnut milk, sunflower milk, whole grain milk, plant milk or a combination thereof

In one aspect, the milk is dairy milk and is sourced from a cow. Further in this aspect, the cow has not been treated with hormones like recombinant bovine somatotropin (rBST, also known as recombinant bovine growth hormone or rBGH). In another aspect, the milk is nonfat milk. Further in this aspect, cream is added to the nonfat milk and the yogurt that results from fermentation of a mixture of cream and nonfat milk is lowfat yogurt. As used herein, “lowfat yogurt” refers to yogurt having from about 1 to about 5 grams of fat per 8 ounce serving, or has about 1, about 1.5, about 2, about 2.5, about 3, about 3.5, about 4, about 4.5, or about 5 grams of fat per 8 ounce serving, where any value can be a lower and upper end-point of a range (e.g., 1 g to 3 g, 2 g to 4.5 g, etc.).

In one aspect, the milk is pasteurized before fermentation. In one aspect, the bacteria used for fermentation remain alive in the yogurt-based beverage after fermentation. Further in this aspect, the bacteria used for fermentation serve as probiotics. As used herein, a “probiotic” is a microorganism that is believed to confer health benefits when consumed. In a further aspect, a probiotic microorganism can improve the microbial balance of the intestines of the individual who consumes it. In one aspect, the probiotic is a bacterium. In an alternative aspect, the probiotic is a fungus such as, for example, a yeast or a mold.

In one aspect, the probiotic is a yeast such as, for example, an organism selected from the genus Saccharomyces, Debaromyces, Candida, Pichia, or Torulopsis. In an alternative aspect, the probiotic is a mold such as, for example, an organism selected from the genus Aspergillus, Rhizopus, Mucor, or Penicillium. In still another aspect, the probiotic is a bacterium such as, for example, an organism selected from the genus Bifidobacterium, Bacterioides, Clostridium, Fusobacterium, Melissococcus, Propionibacterium, Streptococcus, Enterococcus, Lactococcus, Kocuria, Staphylococcus, Peptostreptococcus, Bacillus, Pediococcus, Micrococcus, Euconostoc, Weissella, Aerococcus, Oenococcus, or Lactobacillus. In still another aspect, the probiotic is a combination of organisms from one or more of the genera listed above.

In another aspect, the probiotic is Aspergillus niger, A. oryzae, Bacillus coagulans, B. lentus, B. licheniformis, B. mesentericus, B. pumilus, B. subtilis, B. natto, Bacteroides amylophilus, Bac. capillosus, Bac. ruminocola, Bac. suis, Bifidobacterium adolescentis, Bif. animalis, Bif. breve, Bif. bifidum, Bif. infantis, Bif. lactis, Bif. longum, Bif. pseudolongum, Bif. thermophilum, Candida pintolepesii, Clostridium butyricum, Enterococcus cremoris, E. diacetylactis, E. faecium, E. intermedius, E. lactis, E. mundtii, E. thermophilus, Escherichia coli, Kluyveromyces fragilis, Lactobacillus acidophilus, L. alimentarius, L. amylovorus, L. crispatus, L. bifidus, L. brevis, L. bulgaricus, L. casei, L. curvatus, L. cellobiosus, L. delbrueckii ss. bulgaricus, L. farciminis, L. fermentum, L. gasseri, L. helveticus, L. lactis, L. plantarum, L. johnsonii, L. reuteri, L. rhamnosus, L. sakei, L. salivarius, Leuconostoc mesenteroides, Pediococcus cereviseae (damnosus), P. acidlactici, P. pentosaceus, Propionibacterium freudenreichii, Prop. shertnanii, Saccharomyces cereviseae, Staphyloccus carnosus, Staph. xylosus, Streptococcus infantarius, Strep. salivarius ss. thermophilus, Strep. thermophilus, Strep. lactis, or any combination thereof.

In one aspect, the probiotic is a combination of Lactobacillus bifidus and Lactobacillus acidophilus. In another aspect, an additional probiotic is also included; in this aspect, the additional probiotic can contain Streptococcus thermophilus, Lactobacillus bulgaricus, and Lactobacillus casei.

As used herein, a “colony forming unit” or “CFU” is a measure of how many bacteria in a sample are capable of forming colonies. In this aspect, microorganisms are cultured and only viable cells are counted as CFUs. In one aspect, it is uncertain if a given colony on a culture plate arose from one cell or a group of several cells, hence the use of CFUs to address the uncertainty. In one aspect, Lactobacillus bifidus is present in an amount ranging from 100,000 CFU to 1,500,000 CFU or from about 250,000 CFU to 1,000,000 CFU per 8 ounces of beverage, or is present at about 100,000, about 150,000, about 200,000, about 250,000, about 300,000, about 400,000, about 500,000, about 600,000, about 700,000, about 750,000, about 800,000, about 900,000, or about 1,000,000 CFU per 8 ounces of beverage, where any value can be a lower and upper end-point of a range. In another aspect, Lactobacillus acidophilus is present in an amount ranging from 250,000 CFU to 1,000,000 CFU per 8 ounces of beverage, or is present at about 250,000, about 300,000, about 400,000, about 500,000, about 600,000, about 700,000, about 750,000, about 800,000, about 900,000, about 1,000,000, about 1,250,000, or about 1,500,000 CFU per 8 ounces of beverage, where any value can be a lower and upper end-point of a range.

In another aspect, the beverages described herein contain cannabinodiol. “Cannabinodiol” as used herein refers to a phytocannabinoid present at low concentrations in Cannabis sativa and/or other Cannabis species. In a further aspect, the cannabinodiol is a non THC (tetrahydrocannabinol) and non-narcotic phytocannabinoid.

In one aspect, the cannabinodiol is extracted from hemp seeds or is present as a hemp seed extract. In another aspect, the cannabinodiol exhibits anti-inflammatory properties. In a further aspect, the cannabinodiol is present in an amount of from 5 mL to 100 mL, or from 10 mL to 50 mL per 8 ounces of beverage, or the beverages contain about 5 mL, 10 mL, about 15 mL, about 20 mL, about 25 mL, about 30 mL, about 35 mL, about 40 mL, about 45 mL, about 50 mL, about 60 mL, about 70 mL, about 80 mL, about 90 mL, or about 100 mL of cannabinodiol per 8 ounce serving, where any value can be a lower and upper end-point of a range.

In another aspect, the beverage further contains a prebiotic. As used herein, “prebiotic” refers to a food ingredient that promotes the growth of beneficial microorganisms (such as, for example, microorganisms consumed as probiotics) in the intestine.

In one aspect, the prebiotic is derived from acacia gum or gum arabic, raw dandelion greens, raw leeks, Jerusalem artichokes, raw jicama, underripe bananas, raw chicory root, raw garlic, onions, agave nectar, raw wheat bran, whole wheat flour, yacon, Echinacea, burdock, globe artichoke, wild yam, mugwort, or raw asparagus. In one aspect, the prebiotic is or contains an inulin. In another aspect, the prebiotic can be a “galactooligosaccharide” or “GOS.”

As used herein, “inulin” refers to a naturally occurring polysaccharide produced by a plant such as, for example, chicory. An inulin is a type of dietary fiber and is classified as a “fructan,” or a polymer of fructose. A “fructooligosaccharide” or “FOS” or “oligofructose” is a fructan with a short chain length. As an example, the degree of polymerization in inulin can be from 10 to 60 or, in some instances, can be up to several thousand fructose units, whereas the degree of polymerization in FOS produced from the chemical degradation of inulin can be from 1 to 14 or from 1 to 7. FOS may improve the flavor or odor of food products; FOS are often used as alternative sweeteners but FOS, like inulin, can serve as a substrate for probiotic microorganisms in the large intestine.

In one aspect, the beverages described herein are “synbiotic.” This term refers to products containing both probiotics and prebiotics and specifically to products wherein the prebiotics chosen selectively favor the growth of the probiotic microorganisms.

In some aspects, the beverages disclosed herein include additional ingredients known in the art. In other aspects, the beverages are 100% natural (i.e., made with 100% natural components) with no artificial colors, dyes, preservatives, sweeteners, or the like.

In one aspect, the additional ingredients are selected from water, polydextrose, milk protein concentrate, vitamin D₃, vitamin A, other vitamins, sodium citrate, malic acid, tapioca starch, corn starch, calcium caseinate, potassium carbonate, vegetable juice, salt or sea salt, carrageenan, whey protein concentrate, fruit juice concentrate, cane sugar or another sweetener, chicory root fiber, fruit pectin, locust bean gum, milk, natural flavors, preservatives, or any combination thereof.

A “sweetener” is a compound, extract, or composition that imparts a sweet taste to a food or beverage. Inclusion of a sweetener may assist in improving the flavor of a food or beverage product. In one aspect, the beverages disclosed herein to not contain any added sweeteners. In another aspect, the beverages disclosed herein contain one or more added sweeteners.

In one aspect, the sweetener is a natural sweetener. Examples of sweeteners useful herein include, but are not limited to, cane sugar, beet sugar, coconut sugar, agave nectar, honey, maple syrup, molasses, barley malt syrup, brown rice syrup, date sugar, xylitol, erythritol, lucuma powder, monk fruit extract, stevia extract, or any combination thereof. In one aspect, the sweetener can add calories or other nutrients such as, for example, fiber to the beverages. In an alternative aspect, the sweetener is a non-caloric sweetener.

A “preservative” is a compound or composition that is added to food and/or beverage products to prevent spoilage and prolong shelf life. In one aspect, a preservative can be an artificial or added preservative, such as, for example, sodium benzoate, benzoic acid, a nitrite, a sulfite, sodium sorbate, potassium sorbate, or a similar compound. In one aspect, added preservatives are not found in natural products. In another aspect, the beverages described herein do not contain any added or artificial preservatives.

In one aspect, the beverages described herein include a natural preservative. “Natural preservatives” as described herein include antioxidants such as, for example, vitamins A, C, and E (often present as “mixed tocopherols”). In another aspect, the natural preservative can be rosemary oil or rosemary leaf extract, lemon juice, cinnamon extract, citric acid, or a combination thereof.

“Natural flavors” are defined by the Code of Federal Regulations as essential oils, oleoresins, essences or extractives, protein hydrolysates, distillates, or any products of roasting, heating, or enzymolysis, which contain the flavoring constituents derived from a spice, fruit or fruit juice, vegetable or vegetable juice, edible yeast, herb, bark, bud, root, leaf, or similar plant material, meat, seafood, poultry, eggs, dairy products, or fermentation products thereof, whose significant function in food is flavoring rather than nutritional. In one aspect, the beverages disclosed herein include one or more added natural flavors. In an alternative aspect, the beverages disclosed herein are free from any added natural flavors other than those already possessed by the ingredients described herein.

In one aspect, the beverages disclosed herein are free of THC and gluten. In another aspect, the beverages are OU-D kosher. In still another aspect, the beverages are good sources of calcium and protein.

In one aspect, the components of the beverages disclosed herein can be admixed in any order using techniques known in the art. “Admixing” or “admixture” refers to a combination of two components together when there is no chemical reaction or physical interaction. The terms “admixing” and “admixture” can also include the chemical interaction or physical interaction between any of the components described herein upon mixing to produce the composition. The components can be admixed alone or in water.

In one aspect, the beverages disclosed herein require refrigeration before and/or after opening. In another aspect, the beverages disclosed herein do not require refrigeration.

In one aspect, provided herein is a beverage containing:

(a) lowfat yogurt;

(b) cannabinodiol in the amount of 10 mL to 50 mL per 8 ounces of beverage;

(c) Lactobacillus bifidus in the amount of 250,000 to 1,000,000 CFU per 8 ounces of beverage; and
(d) Lactobacillus acidophilus in the amount of 250,000 to 1,000,000 CFU per 8 ounces of beverage.

In one aspect, the beverages described herein are useful for fighting or reducing inflammation. “Inflammation” as used herein is part of the body's response to harmful stimuli. In the instance of injury or tissue damage, inflammation is a protective response localized to a specific area (such as, for example, redness and swelling around a burn). In another aspect, inflammation can be chronic inflammation or systemic inflammation. Further in this aspect, chronic or systemic inflammation can contribute to an overall decline in the health of the body and may be associated with diseases or conditions ranging from asthma and chronic ulcers, rheumatoid arthritis and other autoimmune diseases, periodontitis, ulcerative colitis, Crohn's disease, chronic hepatitis, chronic sinusitis, some cancers, atherosclerosis, and more. In a further aspect, consuming the beverages described herein can be useful in fighting inflammation. In another aspect, the probiotics and prebiotics contained in the beverages disclosed herein are also useful in fighting inflammation.

In another aspect, consumption of the beverages can correct or improve cases of dysbiosis. As used herein, “dysbiosis” refers to an imbalance of the microbial population on or inside the body, such as in the intestinal tract. In some aspects, when a subject is in a state of dysbiosis, pathogenic bacteria normally present in low amounts flourish. This can lead to improper digestion, malabsorption of food and/or specific nutrients, and other illnesses and conditions due to competition between pathogenic bacteria and useful bacteria such as, for example, those that produce certain vitamins (e.g., vitamin K) from dietary precursors. In another aspect, accumulation of waste products from pathogenic bacteria is another harmful effect of dysbiosis. Prolonged dysbiosis can result from inappropriate diet, antibiotic exposure, alcohol abuse, and the like. Dysbiosis in the intestinal tract is best known, but a state of dysbiosis can also be found among skin flora (leading to, for example, staph infections such as boils, impetigo, cellulitis, and staphylococcal scalded skin syndrome) and in the vagina (leading to, for example, vaginal yeast infections).

Throughout this application, various publications are referenced. The disclosures of these publications in their entireties are hereby incorporated by reference into this application in order to more fully describe the compounds, compositions, and methods described herein.

Various modifications and variations can be made to the compounds, compositions, and methods described herein. Other aspects of the compounds, compositions, and methods described herein will be apparent from consideration of the specification and practice of the compounds, compositions, and methods disclosed herein. It is intended that the specification and examples be considered as exemplary.

What is claimed:

1. A beverage comprising (a) yogurt, (b) cannabinodiol, and (c) a probiotic, wherein the probiotic comprises Lactobacillus bifidus and Lactobacillus acidophilus.

2. The beverage of claim 1, wherein the yogurt comprises lowfat yogurt.

3. The beverage of claim 1, wherein the cannabinodiol is from 10 mL to 50 mL per 8 ounces of beverage.

4. The beverage of claim 1, wherein the Lactobacillus bifidus is from 250,000 CFU to 1,000,000 CFU per 8 ounces of beverage.

5. The beverage of claim 1, wherein the Lactobacillus acidophilus is from 250,000 CFU to 1,000,000 CFU per 8 ounces of beverage.

6. The beverage of claim 1, wherein the beverage further comprises a prebiotic.

7. The beverage of claim 6, wherein the prebiotic comprises an inulin.

8. The beverage of claim 1, wherein the beverage further comprises an additional probiotic.

9. The beverage of claim 1, wherein the beverage further comprises fruit juice concentrate, cane sugar, chicory root fiber, fruit pectin, locust bean gum, milk, water, polydextrose, milk protein concentrate, vitamin D₃, vitamin A, sodium citrate, malic acid, tapioca starch, corn starch, calcium caseinate, potassium carbonate, vegetable juice, salt, carrageenan, whey protein concentrate, natural flavors, sweeteners, preservatives, or any combination thereof

10. The beverage of claim 1, wherein the beverage comprises:
(a) lowfat yogurt;
(b) cannabinodiol in the amount of 10 mL to 50 mL per 8 ounces of beverage;
(c) Lactobacillus bifidus in the amount of 250,000 CFU to 1,000,000 CFU per 8 ounces of beverage; and
(d) Lactobacillus acidophilus in the amount of 250,000 CFU to 1,000,000 CFU per 8 ounces of beverage.

11. A method for treating or preventing inflammation in a subject comprising consuming the beverage of claim 1.

12. A method for treating or preventing dysbiosis in a subject comprising consuming the beverage of claim 1.
